SEQUENCE LISTING (13 sequences)

SEQ ID NO: 1 - protein, 549 aa, Homo sapiens
MPASAPPRRPRPPPPSLSLLLVLLGLGGRRLRAEPGDGAQTWARVSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVSQLTKPISSLTKPYH

SEQ ID NO: 2 - protein, 550 aa, Mus musculus
MLARAPPRRPPRLVLLRLLLLHLLLLALRARCLSAEPGQGAQTWARFARAPAPEAAGLLHDTFPDGFLWAVGSAAYQTEGGWRQHGKGASIWDTFTHHSGAAPSDSPIVVAPSGAPSPPLSSTGDVASDSYNNVYRDTEGLRELGVTHYRFSISWARVLPNGTAGTPNREGLRYYRRLLERLRELGVQPVVTLYHWDLPQRLQDTYGGWANRALADHFRDYAELCFRHFGGQVKYWITIDNPYVVAWHGYATGRLAPGVRGSSRLGYLVAHNLLLAHAKVWHLYNTSFRPTQGGRVSIALSSHWINPRRMTDYNIRECQKSLDFVLGWFAKPIFIDGDYPESMKNNLSSLLPDFTESEKRLIRGTADFFALSFGPTLSFQLLDPNMKFRQLESPNLRQLLSWIDLEYNHPPIFIVENGWFVSGTTKRDDAKYMYYLKKFIMETLKAIRLDGVDVIGYTAWSLMDGFEWHRGYSIRRGLFYVDFLSQDKELLPKSSALFYQKLIEDNGFPPLPENQPLEGTFPCDFAWGVVDNYVQVSPLTKPSVGLLLPH

SEQ ID NO: 3 - protein, 15 aa, Homo sapiens
SQLTKPISSLTKPYH

SEQ ID NO: 4 - protein, 14 aa, Mus musculus
SPLTKPSVGLLLPH

SEQ ID NO: 5 - protein, 15 aa, Artificial
  peptide from mammal secreted Klotho protein
  MISC_FEATURE (2)..(2): X is an amino acid selected from P and Q
  MISC_FEATURE (7)..(7): X is an amino acid selected from S and I
  MISC_FEATURE (8)..(8): X is an amino acid selected from V and S
  MISC_FEATURE (9)..(9): X is an amino acid selected from G and S
  MISC_FEATURE (11)..(11): X is an amino acid selected from L and T
  MISC_FEATURE (12)..(12): X is an amino acid selected from L and K
  MISC_FEATURE (14)..(14): X is an amino acid selected from H and Y
SXLTKPXXXLXXPXH

SEQ ID NO: 6 - DNA, 1650 bp, Homo sapiens
atgcccgccagcgccccgccgcgccgcccgcggccgccgccgccgtcgctgtcgctgctgctggtgctgctgggcctgggcggccgccgcctgcgtgcggagccgggcgacggcgcgcagacctgggcccgtgtctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggacacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttccctgtgactttgcttggggagttgttgacaactacattcaagtaagtcagctgacaaaaccaatcagcagtctcaccaagccctatcactag

SEQ ID NO: 7 - DNA, 1653 bp, Mus musculus
atgctagcccgcgcccctcctcgccgcccgccgcggctggtgctgctccgtttgctgttgctgcatctgctgctgctcgccctgcgcgcccgctgcctgagcgctgagccgggtcagggcgcgcagacctgggctcgcttcgcgcgcgctcctgccccagaggccgctggcctcctccacgacaccttccccgacggtttcctctgggcggtaggcagcgccgcctatcagaccgagggcggctggcgacagcacggcaaaggcgcgtccatctgggacactttcacccatcactctggggcggccccgtccgactccccgatcgtcgtggcgccgtcgggtgccccgtcgcctcccctgtcctccactggagatgtggccagcgatagttacaacaacgtctaccgcgacacagaggggctgcgcgaactgggggtcacccactaccgcttctccatatcgtgggcgcgggtgctccccaatggcaccgcgggcactcccaaccgcgaggggctgcgctactaccggcggctgctggagcggctgcgggagctgggcgtgcagccggtggttaccctgtaccattgggacctgccacagcgcctgcaggacacctatggcggatgggccaatcgcgccctggccgaccatttcagggattatgccgagctctgcttccgccacttcggtggtcaggtcaagtactggatcaccattgacaacccctacgtggtggcctggcacgggtatgccaccgggcgcctggccccgggcgtgaggggcagctccaggctcgggtacctggttgcccacaacctacttttggctcatgccaaagtctggcatctctacaacacctctttccgccccacacagggaggccgggtgtctatcgccttaagctcccattggatcaatcctcgaagaatgactgactataatatcagagaatgccagaagtctcttgactttgtgctaggctggtttgccaaacccatatttattgatggcgactacccagagagtatgaagaacaacctctcgtctcttctgcctgattttactgaatctgagaagaggctcatcagaggaactgctgacttttttgctctctccttcggaccaaccttgagctttcagctattggaccctaacatgaagttccgccaattggagtctcccaacctgaggcagcttctgtcttggatagatctggaatataaccaccctccaatatttattgtggaaaatggctggtttgtctcgggaaccaccaaaagggatgatgccaaatatatgtattatctcaagaagttcataatggaaaccttaaaagcaatcagactggatggggtcgacgtcattgggtacaccgcgtggtcgctcatggacggtttcgagtggcataggggctacagcatccggcgaggactcttctacgttgactttctgagtcaggacaaggagctgttgccaaagtcttcggccttgttctaccaaaagctgatagaggacaatggctttcctcctttacctgaaaaccagccccttgaagggacatttccctgtgactttgcttggggagttgttgacaactacgttcaagtaagtcctttgacaaaacccagtgtcggcctcttgcttcctcactaa

SEQ ID NO: 8 - DNA, 20 bp, Artificial
  Forward primer
tggctttcctcctttacctg

SEQ ID NO: 9 - DNA, 18 bp, Artificial
  Reverse primer
gccgacactgggttttgt

SEQ ID NO: 10 - DNA, 20 bp, Artificial
  Forward primer
ttcaaacccggaagtctttg

SEQ ID NO: 11 - DNA, 20 bp, Artificial
  Reverse primer
ccaggcagacgttcacatta

SEQ ID NO: 12 - DNA, 20 bp, Artificial
  Forward primer
atgggtacaagcgcgtcctg

SEQ ID NO: 13 - DNA, 21 bp, Artificial
  Reverse primer
agccgcaaatgcagatggatc

(19) United States
(12) Patent Application Publication
(10) Pub. No.: US 2018/0086844 A1
(43) Pub. Date: Mar. 29, 2018

(54) ANTIBODIES AGAINST MAMMAL SECRETED KLOTHO PROTEIN

(71) Applicants: UNIVERSITAT AUTONOMA DE BARCELONA, Bellaterra (Cerdanyola del Valles) (ES); FUNDACIÓ INSTITUCIÓ CATALÀ DE RECERCA I ESTUDIS AVANÇATS, Barcelona (ES)

(72) Inventors: Miguel CHILLON RODRIGUEZ, Sant Cugat del Vallès (Barcelona) (ES); Anna MASSO CHACON, Vilanova i la Geltrú (ES); Assumpcio BOSCH MERINO, Sant Cugat del Vallès (Barcelona) (ES)

(21) Appl. No.: 15/560,096

(22) PCT Filed: Mar. 18, 2016
(86) PCT No.: PCT/EP2016/055953

§ 371 Date: Sep. 20, 2017

(30) Foreign Application Priority Data
Mar. 20, 2015 (EP) 15160093.9

(51) Int. Cl. C07K 16/40; G01N 33/68; G01N 33/573

(52) U.S. Cl. CPC C07K 16/40; G01N 33/6857; G01N 33/573; C07K 2317/34; G01N 2800/285; C07K 2317/54; C07K 2317/55; G01N 2800/2821; G01N 2800/2835; C07K 2317/569

(57) ABSTRACT

Antibodies against mammal secreted Klotho protein The invention relates to new antibodies raised against mammal secret isoform of Klotho protein. There are disclosed antibodies against murine and human Klotho proteins, as well as kits and compositions comprising them. The invention also relates to the use of these antibodies as research tools in biochemical and molecular biology assays.

The invention relates to the field of biochemical and molecular biology tools for detecting and quantifying proteins in isolated samples of subjects or in biochemical research assays. In particular it relates to detection and quantification of the mammal Klotho protein.

### BACKGROUND ART

Klotho is a protein not expressed ubiquitously, but detected primarily in the distal convoluted tubule of the kidney, parathyroid hormone-secreting cells and choroid plexus epithelium of the brain. To a lesser extent α-klotho gene is also expressed in the heart, skeletal muscle, urinary bladder, placenta, pancreas, testes, ovaries, colon and inner ear. Of note, the expression of α-klotho is influenced by various physiological and pathological factors. Several studies in mice have revealed that the mutation of the single gene α-klotho on chromosome 13, induces a process of accelerated aging (See. Kuro-o, et al., Mutation of the mouse klotho gene leads to a syndrome resembling ageing. Nature-1997, vol. no. 390, pp.:45-51; and Wang, Y. et al, “Current understanding of klotho”, Ageing Res Rev-2009, vol. no. 8, pp.:43-511, 2) with mimicking typical symptoms such as atherosclerosis, osteoporosis/osteopenia, ectopic calcification in various tissues, emphysema, cognitive problems and infertility. Moreover, knockout α-klotho (kl⁻/kl⁻) mice show dramatically reduced survival with prematurely around 2 months of age. In contrast, transgenic mice overexpressing this gene had between 30 to 40% greater life expectancy. Similarly, several studies indicate that human klotho gene polymorphisms also affect the longevity and appearance of disorders associated with aging.

In mice and humans, α-klotho gene encodes a transcript of a 5.2 kb. In the third exon there is an alternative splicing donor site that can generate two different transcripts: one encoding a transmembrane form (full transcript or full-length, 1014 amino acids) and the other a secreted form of the protein (half transmembrane transcript, 550 amino acids). The full-length transcript encodes a single pass transmembrane protein with a molecular weight of approximately 130 kDa (m-KL). The protein contains three domains: a short transmembrane domain at the C-terminal, an extracellular domain composed of two internal repeated sequences of about 550 amino acids called KL1 and KL2 respectively, and a very short intracellular domain of 10 amino acids. The transcript from alternative splicing generates a truncated form of the protein (s-KL) that is formed solely by the KL1 domain, with an approximate weight of 70 kDa. This alternative mRNA includes a specific secretion signal consisting of 15 amino acid tail that is not found in the m-KL transcript, and for this reason is also called the secreted isoform of Klotho, s-KL (See Matsumura et al., “Identification of the human klotho gene and its two transcripts encoding membrane and secreted Klotho protein”, Biochem Biophys Res Commun-1998, vol. No. 242, pp.:626-630). However, there is some controversy regarding this protein (s-KL) because it has not been detected in body fluids using antibodies. It has been specifically detected at mRNA levels, but not at protein level since the available antibodies cannot differentiate it from the KL1 domain cleaved from the transmembrane Klotho.

In addition, the extracellular domain of the transmembrane form can be cleaved by metalloproteinases ADAM10 and ADAM17 resulting in another form of soluble Klotho of about 130 kDa, which has been detected in serum, urine, and cerebrospinal fluid. Moreover, two recent studies indicate that there is a second recognition site for the proteases ADAM10 and 17 located between the KL1 and KL2 domains, which generates two new 70 kDa isoforms, one contained the KL1 domain only (like the one generated from alternative splicing but without the specific amino acid tail), and the other one contained the KL2 domain (Chen, C. D., et al., “Identification of cleavage sites leading to the shed form of the anti-aging protein klotho”, Biochemistry-2014, vol. No. 53, pp.:5579-5587). Thus, Klotho protein might enter the circulatory system through two main mechanisms: (a) from an alternative splicing (s-KL), and (b) by proteolytic cleavage mediated by ADAM 10 and 17. However, it is unknown the percentage that each of these events occurs.

Examples of these antibodies for recognizing Klotho can be seen from the document Kato et al. “Establishment of the Anti-Klotho Monoclonal Antibodies and Detection of Klotho Proteins in Kidney”, Biochemical and Biophysical Research Communications-2000, vol. no. 267, pp.: 597-602. This document discloses three monoclonal antibodies, named KM2076 (rat IgG2a), KM2119 (rat IgG2b) and KM2365 (mouse IgG1). KM2076 was established by immunizing rats with a part of human KL1 (amino acids 55-261). KM2119 was established by immunizing rats with human KL2 (amino acids 801-954). KM2365 was established by immunizing mice with a synthetic peptide present in any of the mouse Klotho variants (transmembrane and secreted form). Kato et al. indicate that KM2076 and KM2119 recognized in Western blots the 130 kDa protein in mouse and human kidney membrane fractions. To detect the human Klotho protein, the sandwich type ELISA system with KM2076 and KM2365 was established. KM2076 and KM2116 specifically gave a positive staining by immunohistochemical staining. Although these are very sensitive antibodies, they cannot be used for distinguishing the secreted (splice variant) from the different transmembrane forms of the protein.

Several studies have shown that the transmembrane and soluble forms of Klotho may have different functions. Thus, m-KL forms a constitutive binary complex with the FGF-receptor (fibroblast growth factor) and acts as a co-receptor of FGF-23 to maintain phosphate and calcium homeostasis. On the other hand, soluble forms of Klotho have pleiotropic functions such as inhibition of the insulin and IGF1 signalling; inhibition of mTOR-mediated signalling; suppression of oxidative stress; regulation of nitric oxide production and protection against endothelial dysfunction; regulation of the activity of ion channels; oligodendrocyte differentiation and maturation; suppression of the Wnt-mediated signalling; tumor suppression; and also a role in memory formation and cognitive functions.

In the brain, α-klotho is expressed in the ependymal cells and in Purkinje EC cells of the choroid plexus, as well as in hippocampal neurons, although it has been recently demonstrated that α-klotho mRNA and protein are detected throughout the brain parenchyma, co-localizing in neurons and oligodendrocytes during early postnatal development. In addition, recent studies conducted in three independent human cohorts showed that carriers of the Klotho KL-VS allele, which increases secretion of Klotho in vitro, obtained better results in various cognitive tests, including verbal capacity, executive function, visual-spatial processing, and learning. A meta-analysis of the KL-VS variant indicated that it is associated with healthy aging. Finally, recent studies also suggest an anti-inflammatory role in the brain, and second, that Klotho might prevent the development of AD associated with aging probably by inhibiting insulin/IGF-1 signalling and consequently oxidative injury in the brain in a murine model of Alzheimer's disease (AD).

Albeit the high data from Klotho expression and of possible functions, there are no enough tools for discerning the precise role of all its variants, being either the role of the transmembrane form (m-KL) of the protein or those roles of soluble circulating forms, these latter present due to splicing secreted variant (s-KL) or due to the proteolytic cleavage of the transmembrane form (KL1, KL2, KL1-KL2).

### SUMMARY OF THE INVENTION

As above exposed, α-klotho gene encodes two transcripts, one generating a 130 kDa transmembrane protein, and after alternative splicing, a second 70 kDa secreted protein. The transmembrane protein can be cleaved by the metalloproteinases ADAM10 and 17, to generate three other Klotho isoforms (p-KL1, p-KL2 and p-KL1-KL2 (KL1+KL2 domains)), all of them soluble. This variety of Klotho isoforms has led to confusing nomenclature and has made difficult to associate physiological roles to the different Klotho isoforms. To address this, in this description m-KL stands for the full-length, transmembrane form; p-KL stands for proteolyzed Klotho, which is generated by cleavage at the short transmembrane domain (that is, p-KL1-KL2=p-KL for abbreviation purposes); and s-KL stands for secreted Klotho, which is generated by the alternative splicing.

In order to determine the relative contribution of p-KL and s-KL to the levels of circulating Klotho, which is nowadays not known, the inventors provide antibodies as specific tools for detecting s-KL variant of the mammal proteins. These antibodies are highly specific and sensitive in Western Blot analysis of s-KL, therefore allowing differentiation of qualitative and quantitative presence of the soluble Klotho isoforms in a test sample. In particular they recognize in a differential mode s-KL from p-KL and they also suppose in addition a long-felt need.

Thus, in a first aspect the invention relates to antibodies that specifically bind to a peptide amino acid sequence within a mammal secreted Klotho protein (s-KL), said amino acid sequence comprised in the last twenty amino acids of the C-terminal end of the mammal s-KL.

The antibodies of the invention are directed to a peptide that is not present in the m-KL variant. They are directed to a peptide amino acid sequence of the C-terminal end of mammal s-KL, which peptide amino acid sequence is partially codified in an exon and partially in an intron part of the klotho gene.

It is to be noted that this amino acid sequence comprised in the last twenty amino acids of the C-terminal end of the mammal s-KL, particularly of mouse and human, is not coincident with any of the last twenty amino acids of the C-terminal end of protein isoform m-KL and the proteolyzed p-KL1 isoform, this later with a similar molecular weight (70 kDa).

Surprisingly, these highly sensitive and specific antibodies can be reached from immunization with a peptide from 14 to 20 amino acids and comprised in the last twenty amino acids of the C-terminus of s-KL, being said peptide of small length but able to give rise to a pool of antibodies (in case of polyclonal antibodies) or to monoclonal antibodies with low or no cross-reactivity with other epitopes of other antigens.

Another aspect of the invention is a fragment of an antibody as defined above, said fragment being selected from the group consisting of Fab, F(ab′), F(ab′)2, scFv, di-scFv, and sdAb. Indeed, there are included all those fragments of the antibodies of the first aspect of the invention comprising the variable regions of the heavy and/or light chains of the immunoglobulins that, in an antigen-antibody reaction, face the epitope defined by a peptide sequence comprised in the last twenty amino acids of the C-terminal of a mammal s-KL.

As a general rule, antibodies and fragments thereof are usually provided accompanied by carriers (buffered solutions) and preservatives (azide salts, glycerol, etc.). Thus, other aspects of the invention are compositions comprising an antibody as defined above or a fragment thereof, together with carriers and/or preservatives.

These antibodies or fragments, alone or in a composition, may form part of kits for performing the analysis of Klotho protein expression in isolated samples. Therefore, kits for detecting and/or quantifying proteins are also aspects of the invention, said kits comprising an antibody as defined above, or a fragment of said antibody, together with instructions for performing said protein detection.

All these antibodies and fragments may thus be used as research tools, allowing discerning the different roles of p-KL and s-KL isoforms and, consequently, knowing whether some of the functions currently associated with the proteolyzed p-KL are in fact really associated with the secreted s-KL.

The examples below, using this antibodies against s-KL, demonstrate for the first time that the Klotho transcript produced by the alternative splicing generates a stable protein of the expected size (70 kDa), which moreover can be detected directly in protein extracts from murine tissue. In addition, and as above stated, the antibodies permit the study of the specific role that s-KL may play in different organs through the life of the organism, and also facilitate to distinguish between the functions of the proteolyzed KL1 (p-KL1), or any other of the proteolyzed isoforms, and s-KL.

Thus, another aspect of the invention is the use of an antibody as defined above, or of a fragment of said antibody as biochemical and molecular biology reagent.

It is also another aspect of the invention a non-therapeutic method for producing an antibody as defined above, or for producing a fragment of said antibody as defined above, said method involving the injection of a non-human mammal with a peptide defined by an amino acid sequence comprised in the at least the last twenty amino acids of the C-terminal end within a mammal secreted Klotho protein (s-KL), which peptide is optionally conjugated with an adjuvant and/or protein carrier.

Immunization (or injection; used interchangeably herewith) is to be performed with an isolated peptide that consists of SEQ ID NO: 3 (SQLTKPISSLTKPYH) or of SEQ ID NO: 4 (SPLTKPSVGLLLPH), or a peptide from 14 to 20 amino acids comprising any of SEQ ID NO: 3 or SEQ ID NO: 4.

The invention relates also to said isolated peptides consisting in any of SEQ ID NO: 3 or SEQ ID NO: 4.

Both isolated peptides derive from the last twenty amino acids of a mammal secreted Klotho protein (s-KL). These isolated peptides may be defined by a sequence from 14 to 20 amino acids and comprising SEQ ID NO: 5, which is generically defined by formula (I)

SX¹LTKPX²X³X⁴LX⁵X⁶PX⁷(H)n, (I)

wherein n is an integer ranging from 0 to 1, and H means Histidine (H); X¹ is selected from Proline (P) and Glutamine (Q); X² is selected from Serine (S) and Isoleucine (I); X³ is selected from Valine (V) and Serine (S); X⁴ is selected from Glycine (G) and Serine (S); X⁵ is selected from Leucine (L) and Threonine (T); X⁶ is selected from Leucine (L) and Lysine (K); and X⁷ is selected from Histidine (H) and Tyrosine (Y), being all the amino acid symbols those accepted as for the one-letter code abbreviations.

The invention also relates to a method for producing an antibody as defined above, or for producing a fragment of said antibody as defined above, said method involving the immunization with a peptide defined by an amino acid sequence comprised in the at least the last twenty amino acids of the C-terminal end within a mammal secreted Klotho protein (s-KL), which peptide is optionally conjugated with an adjuvant and/or protein carrier.

Due to the role of Klotho in memory and learning as observed in the kl−/kl− mice model (See. Kuro-o, et al., Mutation of the mouse klotho gene leads to a syndrome resembling ageing. Nature-1997, vol. no. 390, pp.:45-51; and Wang, Y. et al, “Current understanding of klotho”, Ageing Res Rev-2009, vol. no. 8, pp.:43-511, 2), the invention also provides as another aspect, a method for the diagnosis of learning and memory problems associated with aging, and/or with neurodegenerative and/or neuropathological diseases selected from the group consisting of Alzheimer's disease, Parkinson's disease, Huntington's disease and Amyotrophic lateral sclerosis, Dementia with Lewy bodies, Creutzfeldt-Jacob disease, Multiple Sclerosis, and Ataxia telangiectasia, which method comprises determining in an isolated sample of a subject the levels of s-KL by means of an antibody as defined above, or by means of a fragment of said antibody.

It is widely known that all these neurodegenerative and/or neuropathological diseases imply, being cause or consequence, impairment or memory loss (of any type; i.e. spatial, temporal, working memory, short-term memory, semantic memory, etc.). In addition, learning problems of new inputs (such as reading or acquisition of new words) are also features of these diseases affecting many of the brain areas. On the other hand, non-pathological or natural aging (or simply aging), is also associated with decline in many types of memory, but not in semantic memory or general knowledge such as vocabulary definitions, which typically increases or remains steady until the late adulthood. Natural aging, or aging as such, is to be understood in the sense of the present invention as the process of becoming older without suffering any particular disease affecting neurons (particularly brain neurons) more than the natural loss of neurons widely known as taking place while people or animals get older.

A method like this allows indeed fast detection of a likely cause for the loss of memory or for learning problems even in adults that previously had been actively working and learning with the habitual standard skills in the population. Detecting that loss of memory and of learning problems is associated with alterations of the levels of s-KL, in respect of subjects without said problems, may represent an early warning of the onset of any neurodegenerative and/or neuropathological disease. It is known that fast detection of these diseases may change evolution of the same.

On the other hand, detection of memory and learning problems in elder animals or humans, associated with alterations of the levels of s-KL in respect of young subjects, may open research lines in order to clarify the memory and learning processes, and further propose solutions for solving impairment of memory and of learning even in young people and in children.

### BRIEF DESCRIPTION OF THE DRAWINGS

FIG. 1, relating to Example 1 depicts several features of a polyclonal antibody according to the invention (Ab-K113 antibody), and biochemical assays that can be performed with it. In (A) it is shown a Western-blot of the s-KL protein from HEK293 cells transduced with the s-KL cDNA (293 s-KL), and from control transduced with the marker GFP gene (293-GFP); in (B) it appears the result of validation of the Ab-K113 antibody, using murine whole brain (BRAIN) and s-KL transduced HEK293 cells (293 s-KL) in a competitive assay with a immunogenic peptide. In (C) it is shown the detection of the s-KL protein by Western-blot from brain (BRAIN) and kidney (KIDNEY) of 6 months-old animals using the K113 antibody. Actine is detected as a 42 kDa protein and was used as control of the Western assays.

FIG. 2, relating to Example 2, shows the analysis of the s-KL protein by Western-blot. Prefrontal cortex (PfCx), cortex (CX), hippocampus (HC), and cerebellum (CB) of 6 and 18 months old C57Bl/6 mice (YOUNG 6M, AGED 8M). Actine (42 kDa) was used to normalize the amount of protein analysed. Samples were quantified by densitometry using ImageJ software, the public domain, Java-based image processing program developed at the National Institute of Health. For each brain area analysed, Fold-change from 18 months-old mice relative to those obtained from 6 months-old mice are depicted in bar diagrams.

FIG. 3, relating to Example 2, shows in bar graphics the analysis of the expression levels of the transmembrane Klotho (m-KL) and secreted Klotho (s-KL) transcripts by qPCR. In FIG. 3 (A) there are depicted the levels in kidney and whole brain of 18 month-old mice compared with 6 months-old mice. In FIG. 3 (B to E) there are depicted the spatial and temporal analysis of the m-KL and s-KL transcripts in prefrontal cortex (B), cortex (C), hippocampus (D) and cerebellum (E) of 6, 9, 12 and 18 months old animals (6M, 9M, 12M and 18M).

### DETAILED DESCRIPTION OF THE INVENTION

All terms as used herein, unless otherwise stated, shall be understood in their ordinary meaning as known in the art. Other more specific definitions for certain terms as used in the present application are as set forth below and are intended to apply uniformly throughout the description and claims unless an otherwise expressly set out definition provides a broader definition.

The term “mammal secreted Klotho (s-KL)”, relates to the Klotho isoform resulting from an alternative splicing transcript, which contains a peptide signal without a transmembrane domain.

A “biochemical and molecular biology reagent” means a compound or composition that is used in the study of the chemical processes within and relating to living organisms, i.e. in biochemistry, and/or in the study of molecular biology, which is the study of the molecular mechanisms by which genetic information encoded in DNA is able to result in the processes of life. Sometimes, molecular biology is considered as a branch of biochemistry, or biochemistry as a tool with which to investigate and study molecular biology.

In the context of the present invention, “epitope” is understood to mean the part of a peptide type macromolecule (or of an antigen), whose sequence and/or spatial configuration is recognized by the immune system (antibodies, T cells, B. cells).

The “C-terminal region” or “the C-terminus” (also known as the carboxy-terminus, COOH-terminus, C-terminal end or carboxy-terminus, all of them used herewith as interchangeable expressions) refers to the end of a protein or polypeptide terminated by an amino acid with a free carboxy group (—COON). The convention for writing peptide sequences is to put the N-terminus on the left and write the sequence from N- to C-terminus. When the protein is translated from messenger RNA, it is created from N-terminus to C-terminus. Wherein in the present invention it is referred to “C-terminal end of a protein” it relates not only to the last amino acid of the linear sequence, but to a region at the end of this linear sequence comprising the last 20 amino acids of the sequence.

“Sensitivity of an antibody” relates to how much antibody is needed to elicit a reaction when talking about immune response, and to the smallest amount of antigen in a test sample than can be detected by the antibody in an immunoassay. On the other hand, “specificity” relates to the capability of the antibody of reacting to one antigen or to many antigens, being said in the later scenario that the “antibody cross-reacts” with some antigens (or with similar epitopes of the same antigen). Cross-reactivity is also a commonly evaluated parameter for the validation of immune and protein binding based assays such as Enzyme-linked immunosorbent assay (ELISA) and radioimmunoassay (RIA). In this case it is normally quantified by comparing the assays response to a range of similar analytes and expressed as a percentage. In practice, calibration curves are produced using fixed concentration ranges for a selection of related compounds and the midpoints (1050) of the calibration curves are calculated and compared. The figure then provides an estimate of the response of the assay to possible interfering compounds relative to the target analyte. High sensitivities and specificities (low cross-reactivity) are aimed for immunoassays.

“Learning and memory problems associated with neurodegenerative and/or neuropathological diseases” indicates that memory is wholly or partially lost in relation to a memory starting point (not neurodegenerative disease), and implying generally or concurring with learning difficulties. Neurodegeneration is the umbrella term for the progressive loss of structure or function of neurons, including death of neurons. Neurodegeneration can be found in many different levels of neuronal circuitry ranging from molecular to systemic.

“Learning and memory problems associated with aging” or with “natural aging” or with “non-pathological aging” (herewith used as synonyms of aging), indicates that memory is wholly or partially lost in relation to a memory starting point (of young animals or human), and implying generally or concurring with learning difficulties.

The “percentage of homology” between two amino acid sequences is to be understood as the percentage of the sequence positions identical or replaced with other amino acids with lateral chains of similar features (i.e. polar, non-polar, with amino groups, with —SH groups), according to the broadly accepted classifications known by an expert in the field. The “percentage of identity” between two amino acid sequences is to be understood as the percentage of the sequence positions with identical amino acids. The percentage of homology and of identity between sequences may be calculated by means of “sequence alignment”. The sequence alignment may be local or global. In the sense of the present invention the percentage of homology and of identity will be calculated, preferably, over a global alignment, among the entire sequence or an entire active fragment of the sequence. Global alignments are more useful when the sequences are similar and have approximately the same size (long). There are several algorithms available in the state of the art for performing these global alignments. There are also bioinformatics tools using such algorithms to obtain the percentage of identity and homology between sequences. As an example, global alignment between sequences may be performed by means of the well-known GGSEARCH or GLSEARCH software. The identity between two amino acid sequences is preferably determined by using the BLASTP algorithm disclosed in Altschul, S. F., et. al. “Gapped BLAST and PSI-BLAST: a new generation of protein a database search programs”, Nucleic Acids Research—1997, Vol. No. 25, pp.: 3389-3402, and NCBI http://www.ncbi.nlm.nih.gov/BLAST.

The invention relates to an antibody that specifically binds to a peptide amino acid sequence comprised in the last twenty amino acids of the C-terminal end within a mammal secreted Klotho protein (s-KL). This means, in particular, that the antibody specifically binds to a peptide sequence comprised in the last twenty amino acids of the C-terminal, said peptide having a length from 14 to 20 amino acids. More particularly, the peptide has a length selected from 14, 15, 16, 17, 18, 19 and 20 amino acids.

In a particular embodiment, the antibody is the one that binds to the mammal secreted Klotho protein (s-KL) with an amino acid sequence selected from the group consisting of SEQ ID NO: 1 and SEQ ID NO: 2.

In a more particular embodiment, the mammal secreted Klotho protein (s-KL) is the one with the amino acid sequence SEQ ID NO: 1. SEQ ID NO: 1 is the amino acid sequence of the transcript from alternative splicing of α-klotho human gene, comprising the KL1 domain sequence, with an approximate weight of 70 kDa, but with a specific secretion signal consisting of 15 amino acid tail that is not found in the m-KL transcript. α-klotho human gene is the one located in Chromosome 13 NC_000013.11 (33016063..33066145) of the assembly GRCh38 (24.12.2013) for the human genome maintained by the Genome Reference Consortium. SEQ ID NO: 1 derives from the corresponding cDNA of SEQ ID NO: 6, deriving from the alternative splicing transcript of the mRNA sequence with the GenBank database accession number NM_004795 of 5012 base pairs, version 3 of 03. May 2014.

In another particular embodiment, optionally in combination with any embodiment above or below, the peptide amino acid sequence comprised in the last twenty amino acids of the C-terminal end is SEQ ID NO: 3 (SQLTKPISSLTKPYH). SEQ ID NO: 3 corresponds, indeed, to the last 15 amino acids of human s-KL of SEQ ID NO: 1. Therefore, it is a peptide sequence within the last twenty amino acids of the C-terminal end of SEQ ID NO: 1.

In another particular embodiment, the antibody or fragment thereof according to the invention binds a peptide amino acid sequence comprised in the last twenty amino acids of the C-terminal of a mammal secreted Klotho protein (5-KL), said s-KL with the amino acid sequence SEQ ID NO: 2. In other words, the mammal secreted Klotho protein (s-KL) consists in SEQ ID NO: 2. SEQ ID NO: 2 is the amino acid sequence of the transcript from alternative splicing of α-klotho mouse gene, comprising the KL1 domain sequence, with an approximate weight of 70 kDa, but with a specific secretion signal consisting of 15 amino acid tail that is not found in the m-KL transcript. α-klotho mouse gene is the one located in Chromosome 5 (150,952,607-150,993,809) of UCSC Genome Browser on Mouse July 2007 (NCBI37/mm9) Assembly for the mouse genome. SEQ ID NO: 2 derived from the corresponding cDNA of SEQ ID NO: 7, deriving in turn from the alternative splicing transcript of the mRNA sequence with the GenBank database accession number NM_013823 of 5124 base pairs, version 2 of 15. Feb. 2015.

In a particular embodiment, optionally in combination with any embodiment above or below, the peptide amino acid sequence comprised in the last twenty amino acids of the C-terminal end is SEQ ID NO: 4. SEQ ID NO: 4 (SPLTKPSVGLLLPH) corresponds, indeed, to the last 14 amino acids of mouse (Mus musculus) s-KL of SEQ ID NO: 2. Therefore, it is a peptide sequence within the last twenty amino acids of the C-terminal end of SEQ ID NO: 2.

Although detected in human and mouse, it is likely that other mammal have secreted Klotho proteins. Antibodies raised against a peptide with a sequence comprised in the last twenty amino acids of such mammal s-KL could be used for detecting these secreted forms of the protein with the advantage of being antibodies with poor cross-reaction with other epitopes.

In particular SEQ ID NO: 3 and SEQ ID NO: 4 have a percentage of identity of 57% if compared with the Multiple Sequence Alignment ClustalW2 algorithm of European Molecular Biology Laboratory of the European Bioinformatics Institute (EMBL-EBI). On the other hand, the identity is of 83% using BLASTP algorithm disclosed in Altschul, S. F., et. al. (supra).

Thus, antibodies rose against human SEQ ID NO: 3 are useful in the detection of mouse proteins comprising SEQ ID NO: 4, and vice versa.

In another particular embodiment, also optionally in combination with any embodiment above or below, the antibody is a polyclonal antibody.

In another particular embodiment, the antibody is a monoclonal antibody. More particularly the monoclonal antibody is further processed to be a chimeric antibody, a humanized antibody, or a bispecific antibody.

In another particular embodiment the antibody is an affinity matured antibody (produced by affinity matured B cells).

In another particular embodiment the antibody is a polyclonal or monoclonal human antibody.

Considering that antibody-antigen reaction takes place within some fragments of the whole antibody (in particular those variable parts of heavy and light chains of the immunoglobulin), the invention encompasses as particular embodiments the fragments of the antibodies discloses in any of the embodiments and aspects above, said fragments selected from the group consisting of Fab, F(ab′), F(ab′)2, scFv, di-scFv, and sdAb. All these fragments specifically bind to a peptide amino acid sequence within a mammal secreted Klotho protein (s-KL), said peptide sequence comprised in the last twenty amino acids of the C-terminal end of the mammal secreted Klotho protein (s-KL).

All these antibodies are in particular supplied in a composition comprising any one of them together with acceptable carriers and/or preservatives. Examples of carriers and/or preservatives include buffered salt solutions with azide, such as sodium azide, and glycerol. Other carriers are silicone oil emulsions with any of sodium chloride, sucrose, sorbitol, Tween® 20, Tween® 80, and mixtures thereof. The “acceptable carriers and/or preservatives” are those acceptable materials, compositions or vehicles. Each component must be acceptable in the sense of being compatible with the other ingredients of the composition comprising the antibodies. It must also be suitable for use in conjunction with the antibodies while performing its function, in particular in the case of preservatives that are compounds avoiding contamination of the composition by microorganisms.

The kits for detecting and/or quantifying proteins comprising an antibody as defined above, or of a fragment of said antibody contain, in a particular embodiment, instructions in paper or in an electronic data carrier, such as in a CD ROM or a semiconductor ROM, or a magnetic recording medium, for example a floppy disc or hard disk.

In another particular embodiment of the kits according to the invention, they are for carrying out biochemistry assays and/or molecular biology techniques selected from the group consisting of enzyme immunoassays, radioimmunoassay, immunoprecipitation assays, immunohistochemistry assays, western blots and eastern blots.

As above exposed, the antibodies or any fragment thereof can be used as biochemical and molecular biology reagents. Particularly, they are used as reagents for techniques of molecular biology, most in particular for performing western-blots and/or eastern-blots. As biochemical reagents, they can be used in enzyme immunoassays, radioimmunoassay, immunoprecipitation assays, or immunohistochemistry assays. Examples of enzyme immunoassays include ELISA.

The invention encompasses also a non-therapeutic method for producing an antibody as defined above, or for producing a fragment of said antibody, which method involves (or comprises) the injection of a non-human mammal with a peptide comprised in the last twenty amino acids of the C-terminal end of a mammal secreted Klotho protein (s-KL), said peptide being optionally conjugated (which means linked by ionic or covalent linkages) with an adjuvant and/or protein carrier. A protein carrier is a protein with the ability to stimulate antibody production against peptides and small proteins of 20-30 amino acids, which require aid to elicit a response. Carrier proteins act as transporters of the peptides and allow visualization or exposition to immune system. Non-limiting examples of adjuvants and/or protein carriers include Montanide ISA 50, Freund's adjuvant, the protein carrier Keyhole limpet hemocyanin (KLH), alum, and paraffin oil. All these adjuvants and/or protein carriers improve immune response to the peptide of interest.

It is to be understood that the non-therapeutic method for producing an antibody or non-medical method (used interchangeably) encompasses only a method applied to a non-human mammal, said non-human mammal being a health individual, and which is experimentally and industrially used as a source of antibodies. That is, the non-human mammal is inoculated (injected, or immunized; used interchangeably) with the aim of producing antibodies but excluding any prophylactic and/or therapeutic aim of said non-human mammal. Health individual means that the non-human mammal is not suffering any disease that could be treated with the injected peptide by means of a response of the immune system. Non-limiting examples of non-human mammals include mice, rabbits, goats, and dogs.

In a particular embodiment of the method, optionally in combination with embodiments above or below, the peptide amino acid sequence for injection is an amino acid sequence from 14 to 20 amino acids length and comprised in the last twenty amino acids of the C-terminal end of a mammal secreted Klotho protein. Most in particular the length of the peptide is from 14 to 16 amino acids, and even most particularly they are the last 14, 15 or 16 amino acids of a mammal s-KL protein.

In a particular embodiment of this method, the peptide for injection is selected from SEQ ID NO: 3 and SEQ ID NO: 4.

Injection is to be performed with an isolated peptide that consists of SEQ ID NO: 3 (SQLTKPISSLTKPYH) or of SEQ ID NO: 4 (SPLTKPSVGLLLPH), or with a peptide from 14 to 20 amino acids comprising any of SEQ ID NO: 3 or SEQ ID NO: 4.

In another particular embodiment, the method is for obtaining a monoclonal antibody, where a hybridoma cell line is grown in a suitable medium and the antibody is recovered from said medium.

The invention encompasses also a method for the diagnosis of learning and memory problems associated with aging, and/or with neurodegenerative and/or neuropathological diseases, which methods comprise determining in an isolated sample of a subject, the levels of s-KL by means of an antibody as defined above, or by means of a fragment of said antibody. Preferred diagnosis methods comprise also determining other isoforms m-KL, p-KL of the protein, thus leading to an accurate expression pattern of this protein involved in diseases related to memory loss, and to learning difficulties associated with aging, and/or with neurodegenerative and/or neuropathological diseases selected from the group consisting of Alzheimer's disease, Parkinson's disease, Huntington's disease and Amyotrophic lateral sclerosis, Dementia with Lewy bodies, Creutzfeldt-Jacob disease, Multiple Sclerosis, and Ataxia telangiectasia. In a particular embodiment, the method is for the diagnosis of Alzheimer's disease.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### EXAMPLES

In order to exemplify some of the assays in which the antibodies of the invention may be employed, there are exposed below some analysis of the expression of Klotho protein performed in mouse.

All these examples allow concluding that antibodies according to the invention permit the study of the specific role that s-KL may play in different organs through the life of the organism, and also facilitate to distinguish between the functions of the proteolyzed KL1 and s-KL.

### Example 1. Generation of a Specific Antibody Against the Mouse s-KL Protein

Currently, the available anti-Klotho antibodies recognize epitopes that are common for both, the secreted and the transmembrane Klotho proteins (including also the soluble-cleaved Klotho; p-KL). To specifically analyse the protein levels of the secreted Klotho protein (s-KL), an antibody against the exclusive peptide (15 amino acids for murine s-KL, and 16 amino acids for human s-KL) at the C-term of the s-KL protein was generated.

First, by using different immunogenicity sequence analysis (Chon&Fasman Beta-Turn Prediction, Kolaskar&Tongaonkar Prediction, Antigenicity Parker Hydrophilicity Prediction and Emini Surface Accessibility Prediction) a good antigenicity was predicted for the SPLTKPSVGLLLPH (SEQ ID NO: 4) peptide. It was then blasted to search for other proteins containing this epitope. Only the secreted Klotho protein had a 100% of identity.

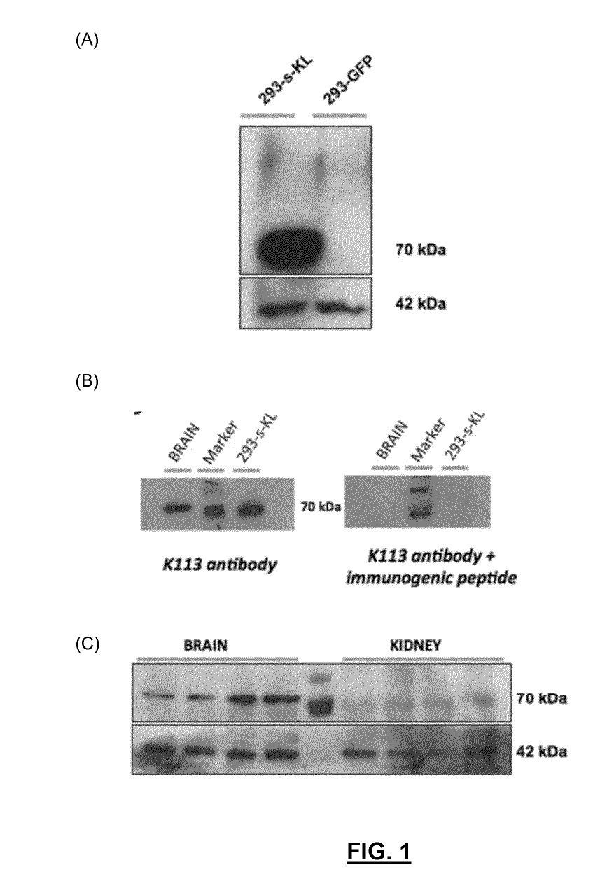

A specific polyclonal antibody (herewith named Ab K113) against s-KL was generated in rabbit by EZBiolab company (Carmel, USA) using the designed immunogenic peptide of SEQ ID NO: 4 as antigen. FIG. 1A, which corresponds to the image of a Western-Blot of the s-KL protein from HEK293 cells transduced with the s-KL cDNA, shows that the K113 antibody detected a band of approximately 70 kDa in these HEK293 cells transduced with the s-KL cDNA, but not in HEK293 cells transduced with the GFP gene (Control). The expected size of s-KL is 62.3 kDa. It is noteworthy that single meaningful bands were detected with the polyclonal K133, thus illustrating its high specificity.

Cell culture and transfection of the cells was performed as follows: HEK-293 (QBiogene) were cultured in six-well plates with DEMEM (Lonza)+10% (v/v) FBS (Biowest), at 37° C. and 5% CO2. Transfections were performed in 60-70% confluent cultures with DMEM+1% (v/v) FBS, using 6 μg of s-KL or GFP plasmid DNA complexed with PEI-25 KDa (Aldrich) per 10⁶ cells. Cells were harvest 48 hours after transfection and mRNA and proteins extracted.

The K113 antibody was further validated by competition with the SPLTKPSVGLLLPH (SEQ ID NO: 4) peptide in s-KL-transduced HEK293 cells as well as in brain tissue (FIG. 1B). Western blot images of FIG. 1B show that competition with the peptide of SEQ ID NO: 4 retained antibody K113 from reaction with the assayed tissues, thus confirming specificity of the same.

Moreover, in an assay with brain and kidney tissue, the protein levels of s-KL were 9-fold higher in whole brain than in kidney (FIG. 1C) of 6 months-old mice, despite in a parallel assay with the same samples quantification cycles values (Cq) in the quantitative PCR (qPCR) of s-KL levels in brain and kidney were similar by densitometry.

This example and also those herein below show how the antibodies of the invention can advantageously be used in Western blots as single primary antibodies. In addition, they are highly specific showing single meaningful bands.

For all these assays and those of the examples following, animal protocols were reviewed and approved by the Ethical Committee of the Universitat Autonoma de Barcelona. All experimental procedures were performed in the Central Animal Facilities of the Universitat Autonoma de Barcelona, according to the current national regulations. Eight to ten weeks-old 057131/6 mice were used per group. At specific given times, mice were euthanized and samples from kidney and different brain areas (prefrontal cortex, cerebral cortex, hippocampus and cerebellum) were collected and transferred to tubes, snap frozen in liquid nitrogen and stored at −80° C. until RNA or protein extraction.

For the analysis of mRNA levels (qPCR), used as control of expression and as comparative example, total RNA was obtained from brain/kidney samples and extracted from the tissue samples using the QIAzol Lysis Reagent (Qiagen), then quantified on a NanoDrop spectrophotometer (Thermo Scientific) and reverse transcribed into cDNA with iScript cDNA Synthesis Kit (Bio-Rad). Gene-specific primers used for the qPCR analysis were: s-KL Fwd: 5′-TGGCTTTCCTCCTTTACCTG-3′ (SEQ ID NO: 8); s-KL Rv: 5′-GCCGACACTGGGTTTTGT-3′ (SEQ ID NO: 9); m-KL Fwd: 5′-TTCAAACCCGGAAGTCTTTG-3′ (SEQ ID NO: 10); m-KL Rv: 5′-CCAGGCAGACGTTCACATTA-3′ (SEQ ID NO: 11); m36B4 Fwd: 5′-ATGGGTACAAGCGCGTCCTG-3′ (SEQ ID NO: 12); m36B4 Rv: 5′-AGCCGCAAATGCAGATGGATC-3′ (SEQ ID NO: 13). Primers were initially optimized by standard PCR using Taq DNA polymerase (Qiagen) and visualized by ethidium bromide stain on a 2% low melting agarose gel. qPCR was performed on a Bio-Rad CFX-384 PCR machine using iTaq™ Universal SYBR Green Supermix (Bio-Rad). A two-step PCR reaction was carried out as follows: 1 cycle of 98° C. for 2 minutes followed by 40 cycles of 95° C. for 5 seconds and 58° C. for 30 seconds. Samples were run as triplicates. The analysis of qPCR output data followed the manufacturer-suggested ΔCt method. Cycle thresholds (Ct) were measured, and the relative expression of genes was calculated by comparison of Ct values. All samples were normalized to m36B4, used as a reference gene. Melt-curve analysis was used to confirm the production of a single amplicon for each gene tested. Based on the RT-qPCR assay efficiency, gene amplification at a level higher than 35 cycles (ΔCt of 15) was considered to have no expression. A “no template control” was also included in each run.

For the anti-Klotho antibody characterization, samples were submitted to sonication and homogenized in lysis buffer (50 mM Tris-CI pH 7.4, 150 mM NaCl, 1 mM ethylenediamine tetraacetic acid [EDTA], 1% NP-40, 0.25% sodiumdeoxycholate and Complete Mini EDTA-free protease inhibitor cocktail tablets (Roche Diagnostics,). Total protein concentration was determined using the Pierce BCA Protein Assay (Thermo Scientific) according to the manufacturer's instructions and bovine serum albumin (BSA) as the standard. Sample absorbance at 562 nm was measured using a NanoDrop 1000 UV/Vis spectrophotometer (Thermo Scientific). Protein extracts (15-25 μg per sample) were loaded onto denaturing acrylamide gels and further electro-transferred to PDVF membranes (Amersham). Primary antibodies were incubated in the presence of 5% (w/v) skim milk combined with western blotting detection reagent (ECL Plus; Amersham). The Ab K113 rabbit polyclonal antibody was used at a dilution of 1/5000; polyclonal antibody anti-actin (Sigma, A2066) at 1/1000; and secondary polyclonal antibody rabbit HRP-anti-Ig (Dako-Cytomation, P0399) at 1/10000. Band pixel intensities were quantified by Image J. (Wayne Rasband National Institutes of Health, USA) and normalized by anti-actin levels in each lane.

The statistical Analysis was as follows: Data are presented as mean values±SEM. Statistical calculations were performed using the G-Stat version 2.0 and Prism 5.04 for windows statistical programs. Statistical significance between individual groups was determined by Student-t test (unpaired, two-tailed) or one-way ANOVA followed by Tukey post hoc analysis. In all the statistical analyses, P<0.05 was considered significant.

### Example 2. Detection of the Secreted Klotho (s-KL) Protein in Different Brain Areas of Young and Old Adult Mice

In order to show advantages of using the antibodies of the invention as biochemical agents, inventors performed several assays with them. The aim was determining if detection of s-KL with the antibodies could give rise to concluding data about behaviour and possible functions of s-KL.

Therefore, the levels of the s-KL protein were analysed in prefrontal cortex (PfCX s-KL), cortex (CX s-KL), hippocampus (HC s-KL) and cerebellum (CB s-KL) of 6 months old and 18 months-old mice using the K113 antibody.

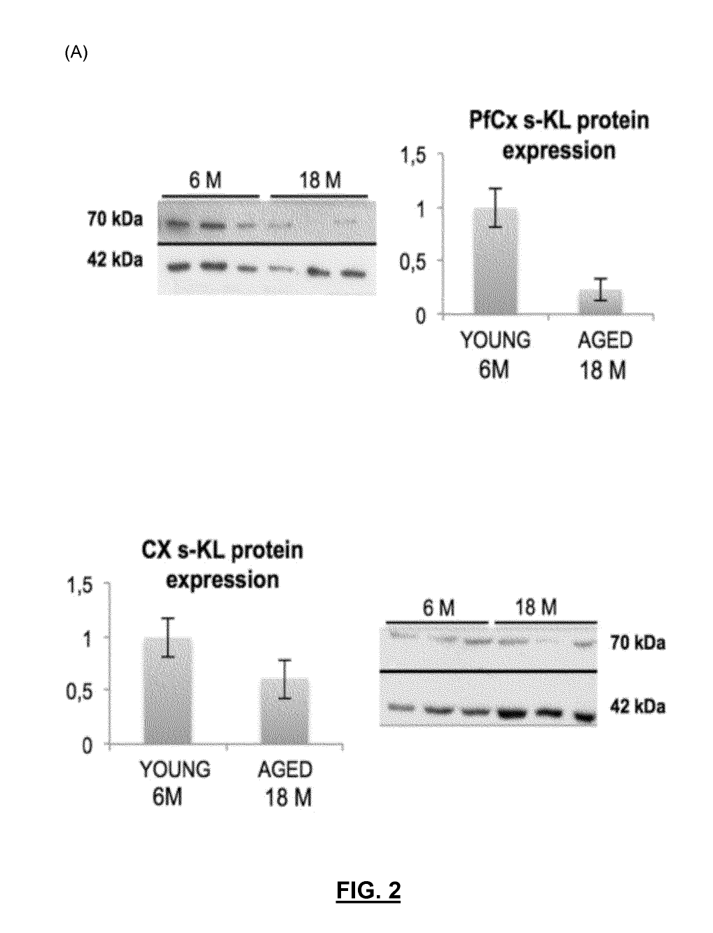

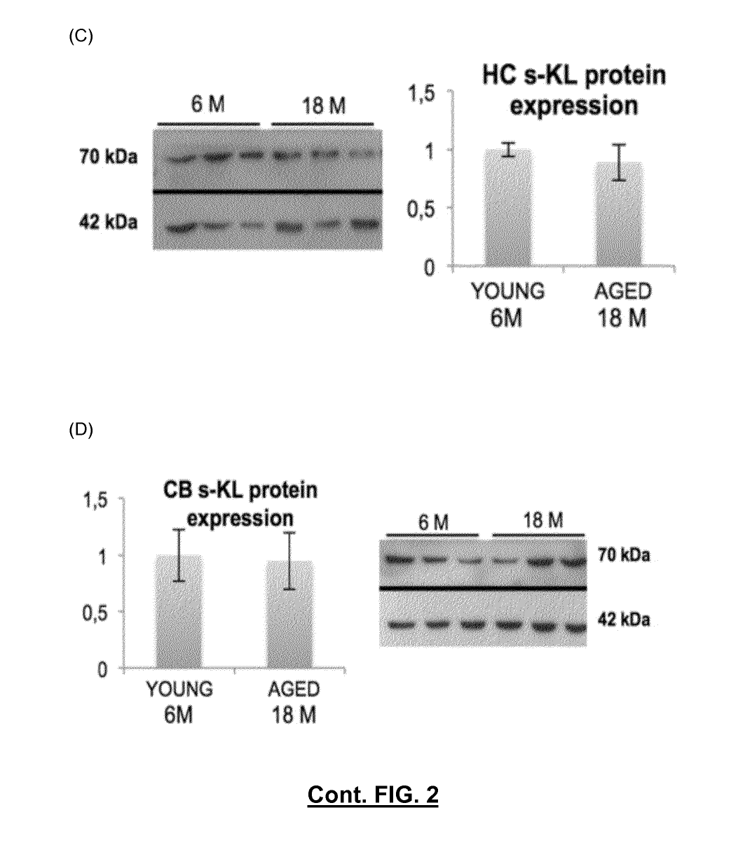

Data are depicted in FIG. 2, wherein Western-blot analysis is shown for each region together with densitometry assays (using Image J software). FIG. 2(A) depicts data from PfCX, FIG. 2(B) data from CX, FIG. 2(C) depicts data from HC and FIG. 2(D) data from CB.

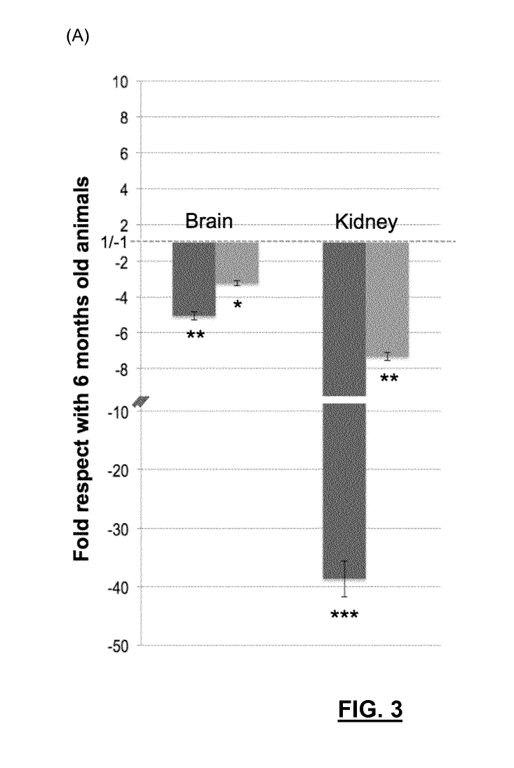

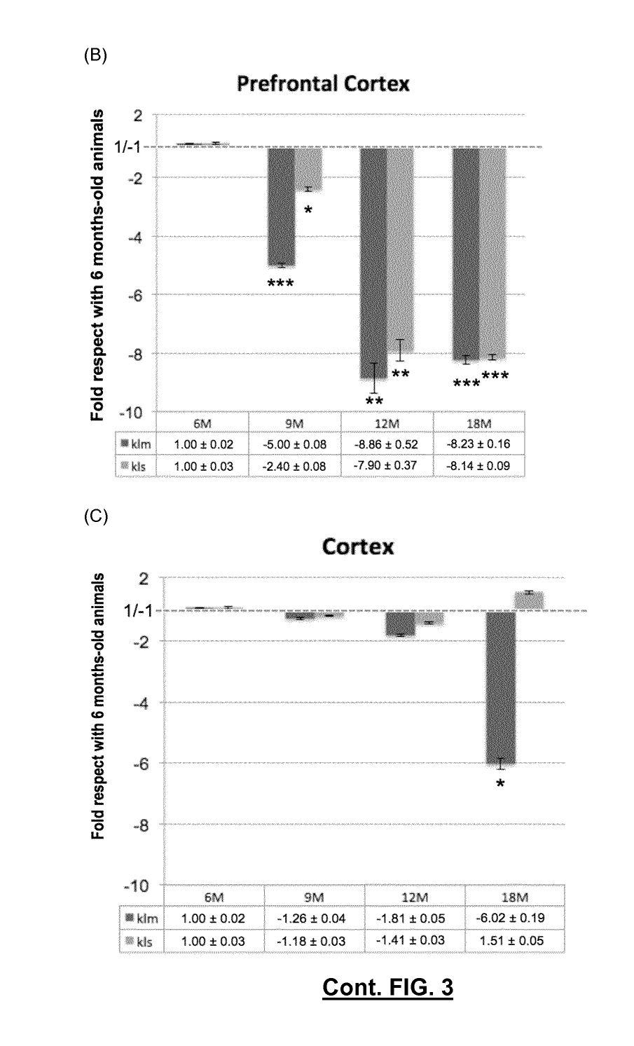

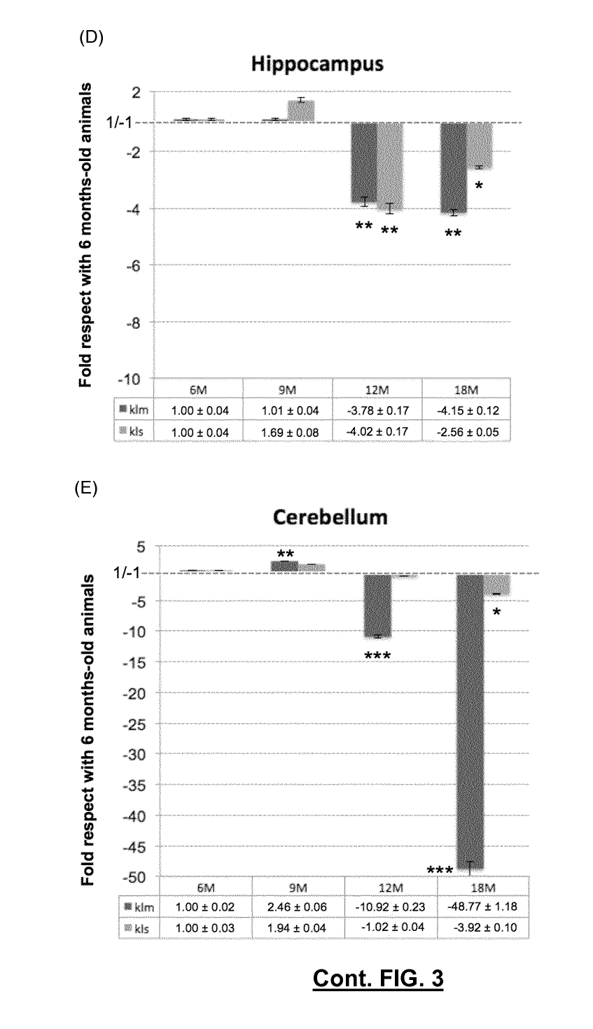

In parallel, the levels of the transcript were also determined by qPCR in the same tissue fractions. These qPCR data are depicted in FIG. 3. Analysis was performed as indicated in example 1 and expression levels calculated with ΔCt method. FIG. 3 (A) shows the expression levels of s-KL and m-KL in whole brain and kidney as the fold respect with six months old animals. Data in table under the bar diagram depicts the represented values. FIG. 3 (B to E) show the expression levels of s-KL and m-KL in different brain areas (prefrontal cortex, cortex, hippocampus and cerebellum) in transgenic animals with 6, 9, 12 and 18 months old (the transgenic was the Alzheimer's disease model known as 3×Tg-AD mice). Again data in tabled under the bar diagrams depict the represented values. Levels are also expressed as the fold respect with the 6 months old animals.

In agreement with the levels of the s-KL mRNA transcript (data from FIG. 3), the levels of the s-KL protein determined by Western blot with Ab K113 declined with aging in the cortex, the prefrontal cortex and hippocampus (down to 49%, 48% and 63% respectively). However, in cerebellum, the s-KL levels were similar at both ages. Indeed, the correlation between mRNA and protein levels in brain areas is difficult since proteins produced in other brain areas may be present because of axonal projections from distant neurons. Of note, this is the first time the levels of the secreted Klotho protein are quantified, without measuring the KL1 or KL1+KL2 domains produced after the cleavage of the transmembrane Klotho protein.

This assay demonstrates that K113 antibody permits the study of the specific role that s-KL may play in different organs through the life of the organism, and it will also facilitate distinguishing between the functions of the proteolyzed KL1 (p-KL) and s-KL.

In this regard, the K113 antibody is a valuable tool, not only for Western-Blots, but also for ELISA assays. For instance, the K113 antibody has shown that although the levels of s-KL (mRNA) in brain and kidney are similar, the s-KL protein is about 10 times more abundant in brain than in kidney. This unexpected difference could suggest that while s-KL produced in kidney might be rapidly secreted to bloodstream, the s-KL produced in brain seems to accumulate within the brain, although other possibilities as that renal s-KL also accumulates in the brain, or that brain-s-KL is able to partially reach bloodstream cannot be discarded.

### Example 3. Moderate Continuous Exercise in Adulthood Prevents the Decline of the s-KL and m-KL Transcripts

Moderate continuous exercise is considered healthy for the organism, and especially in adulthood may help to prevent, among others, muscular and cardiovascular problems. In fact, some authors have hypothesized that the Klotho protein may respond or may be affected by physical activity since Klotho interactions are mediated via growth factors such as IGF-1. Moreover, a recent report also suggests an important link between Klotho deficiency and age-associated muscle deterioration, while aerobic exercise increases plasma Klotho levels. To this end it was analysed whether in the CNS continuous exposure to a healthy lifestyle during aging could affect or maintain the expression levels of the s-KL and m-KL transcripts. Thus, two groups of mice of 6 and 12 months of age were exposed dairy to the running wheel for six months (from 6 to 12 months of age), and their expression profile was compared to control mice of the same age, which did not exercise. Interestingly, in animals with moderate continuous exercise, the s-KL and m-KL expression levels were higher than in control animals (data not shown) suggesting a positive correlationship between healthier status and higher expression levels of s-KL and m-KL. Of note, although this correlationship was observed in young and old animals, this effect was more pronounced in old mice (12 months of age) reaching for example levels of 8 and 17 fold higher expression of s-KL and m-KL respectively in the hippocampus of exercised mice compared to no exercised or sedentary controls. However, the s-KL and m-KL expression profiles were not uniform through the different brain areas analyzed, which indicates not only temporal but also differential spatial regulation in brain of the two klotho transcripts. Interestingly, despite the old mice exercised less than 50% that of young mice (data not shown) the effect, except in cortex, was more significant in older mice.

Although these data derive from qPCR analysis, parallel assay may be done with K113 antibody (polyclonal antibody), specifically detecting s-KL and thus elucidating the role of s-KL protein in the establishment of health status and in the effects in aging processes.

All these examples allow concluding that antibodies raised against a peptide comprised in the last twenty amino acids of the C-terminal end of a mammal s-KL, are high specific tools for detecting only this Klotho protein splice variant. In addition the antibodies confirm that the protein actually exists and may have an important role in the processes involving learning and memory deficiencies due to aging and due to neurodegenerative and/or neuropathological diseases selected from the group consisting of Alzheimer's disease, Parkinson's disease, Huntington's disease and Amyotrophic lateral sclerosis, Dementia with Lewy bodies, Creutzfeldt-Jacob disease, Multiple Sclerosis, and Ataxia telangiectasia.

### REFERENCES CITED IN THE APPLICATION

Kuro-o, et al., “Mutation of the mouse klotho gene leads to a syndrome resembling ageing”. Nature-1997, vol. no. 390, pp.:45-51.
Wang, Y. et al, “Current understanding of klotho”, Ageing Res Rev—2009, vol. no. 8, pp.:43-511
Matsumura et al., “Identification of the human klotho gene and its two transcripts encoding membrane and secreted klotho protein”, Biochem Biophys Res Commun—1998, vol. No. 242, pp.:626-630
Chen, C. D., et al., “Identification of cleavage sites leading to the shed form of the anti-aging protein klotho”, Biochemistry-2014, vol. No. 53, pp.:5579-5587.
Altschul, S. F., et. al. “Gapped BLAST and PSI-BLAST: a new generation of protein a database search programs”, Nucleic Acids Research—1997, Vol. No. 25, pp.: 3389-3402.

1.-16. (canceled)

17. A method for the diagnosis of learning and memory problems associated with aging, and/or with neurodegenerative and/or neuropathological diseases selected from the group consisting of Alzheimer's disease, Parkinson's disease, Huntington's disease and Amyotrophic lateral sclerosis, Dementia with Lewy bodies, Creutzfeldt-Jacob disease, Multiple Sclerosis, and Ataxia telangiectasia, which method comprises determining in an isolated sample of a subject the levels of s-KL by means of an antibody or of a fragment thereof, which specifically bind to a peptide amino acid sequence within a mammal secreted Klotho protein (s-KL), said peptide sequence comprised in the last twenty amino acids of the C-terminal end of the mammal secreted Klotho protein (s-KL).

18. The method according to claim 17, wherein the disease is Alzheimer's disease.

19. The method according to claim 17, wherein the peptide sequence comprised in the last twenty amino acids of the C-terminal end of the mammal secreted Klotho protein (s-KL) has a length from 14 to 20 amino acids.

20. The method according to claim 17, wherein the mammal secreted Klotho protein (s-KL) is the one with an amino acid sequence selected from the group consisting of SEQ ID NO: 1 and SEQ ID NO: 2.

21. The method according to claim 17, wherein the mammal secreted Klotho protein (s-KL) is the one with the amino acid sequence SEQ ID NO: 1.

22. The method according to claim 21, wherein the peptide amino acid sequence comprised in the last twenty amino acids of the C-terminal end is SEQ ID NO: 3 (SQLTKPISSLTKPYH).

23. The method according to claim 17, wherein the mammal secreted Klotho protein (s-KL) is the one with the amino acid sequence SEQ ID NO: 2.

24. The method according to claim 23, wherein the peptide amino acid sequence comprised in the last twenty amino acids of the C-terminal end is SEQ ID NO: 4 (SPLTKPSVGLLLPH).

25. The method according to claim 17, wherein the antibody is a polyclonal antibody.

26. The method according to claim 17, wherein the antibody is a monoclonal antibody.

27. The method according to claim 17, wherein the fragment of the antibody is selected from the group consisting of Fab, F(ab′), F(ab′)2, scFv, di-scFv, and sdAb.

28. The method according to claim 17, wherein said fragment specifically binds to a peptide amino acid sequence within a mammal secreted Klotho protein (s-KL), said peptide sequence comprised in the last twenty amino acids of the C-terminal end of the mammal secreted Klotho protein (s-KL).

29. The method according to claim 17, wherein the antibody or the fragment is conjugated with an adjuvant and/or protein carrier for being used as reagent in biochemistry assays and/or molecular biology techniques selected from the group consisting of enzyme immunoassays, radioimmunoassay, immunoprecipitation assays, immunohistochemistry assays, western blots and eastern blots.
